(19) United States
(12) Patent Application Publication
(10) Pub. No.: US 2009/0306550 A1
(43) Pub. Date: Dec. 10, 2009

(54) Ultrasonic Endometrial Cryoablation Method

(76) Inventor: Eilaz Babaev, Minnetonka, MN (US)

Correspondence Address: Bacoustics, LLC, 5929 BAKER ROAD, SUITE 470, MINNETONKA, MN, 55345, US

(21) Appl. No.: 12/132,643

(22) Filed: Jun. 4, 2008

(51) Int. Cl. A61B 18/02; A61N 7/00

(52) U.S. Cl. 601/2; 606/21

(57) ABSTRACT

The present invention relates to an ultrasound assisted cryogenic surgical instrument, more particularly, to methods and devices utilizing ultrasound energy for treatment of the endometrium to control heavy uterine bleeding (menorrhagia) or other conditions. The device of the present invention comprises an ultrasound generator, an ultrasound transducer, a transducer tip at the distal end of the ultrasound transducer, and a radiation surface. Ultrasonic radiation is directed into the tissue being ablated. A cryogenic solution is circulated through the ultrasound tip to transfer thermal energy away from the tissue to freeze the tissue being ablated by providing a synergistic effect with the ultrasonic radiation.

### BACKGROUND OF THE INVENTION

The present invention relates generally to an ultrasound assisted cryogenic surgical instrument, more particularly, to methods and devices utilizing ultrasound energy for treatment of the endometrium to control heavy uterine bleeding (menorrhagia) or other conditions that may benefit from tissue ablation.

Menorrhagia is a common problem with a variety of causes. Menorrhagia may be due to hormonal disturbances, uterine fibroids, polyps, overgrowth of the uterine lining (hyperplasia), or cancer. Furthermore, medical conditions, such as bleeding disorders or thyroid disease, may also contribute. When no specific anatomical cause is identified of the menorrhagia, or if disturbances do not improve with hormone therapy, endometrial ablation (destruction of the uterine lining) may be an alternative to hysterectomy.

Hysterectomy has, for many years, been the most widely used treatment for menorrhagia. It can be performed abdominally, vaginally or laparoscopically. However, hysterectomy is a major surgical procedure with inherent risks and the potential for complications. Although hysterectomy yields a high level of satisfaction in that it guarantees the permanent cessation of menstrual bleeding, it is a major procedure. Its invasiveness, morbidity, mortality and costs are well-known disadvantages of the procedure. In addition, hysterectomy can lead to a variety of psychological and physical changes in women. For these various reasons, other less invasive treatments have been sought.

Endometrial ablation was adopted as a less invasive alternative to hysterectomy. Endometrial ablation permits preservation of the uterus and reduces uterine bleeding in most patients. Endometrial ablation is less invasive, more convenient and less expensive than hysterectomy, at least when no complicating gynecologic conditions are involved. Women typically prefer endometrial ablation to hysterectomy because the surgery is less invasive, involves less risk of early menopause and sexual impairment, the changes wrought are less profound, and the hospital stay and convalescence are shorter.

Endometrial ablation procedures can be accomplished through a variety of techniques including the application of heat, radiation or freezing.

Heat based ablation techniques include electrocautery and thermal balloon procedures. Electrocautery is a method of endometrial ablation that uses instruments such as a “roller-ball” or wire loop and is performed under anesthesia in the operating room through a hysteroscope. Thermal balloon endometrial ablation is a technique that is performed in an outpatient surgical center or in a doctor's office. A triangular balloon is placed into the uterus and filled with fluid. The fluid in the balloon is then heated for several minutes. During this time, most of the uterine lining is destroyed.

Radiation techniques include microwave and laser ablation. Microwave and laser ablation procedures tend to be painful even with local anesthesia and require highly skilled practitioners to administer the procedures. Microwave ablation is also appropriate only for the well-formed uterus, because microwave endometrial ablation tends to be incomplete in women whose uterine cavity is hypertrophied or highly deformed. Microwave ablation is also painful, because the cervix must be dilated to 9-mm in order to insert the microwave waveguide, and that dilatation process can be painful even under local anesthesia. In addition they carry risk of complications, which include hematometra, infection and internal organ injury.

Freezing of the uterine lining is also useful for endometrial ablation. Cryoablation procedures involve deep tissue freezing which results in tissue destruction due to rupture of cells and/or cell organelles within the tissue. Deep tissue freezing is effected by insertion of a tip of a cryosurgical device into the tissue, either endoscopically or laparoscopically, and a formation of, what is known in the art as, an ice-ball of frozen tissue around the tip.

Endometrial cryoablation is typically performed either by utilizing a single cryoprobe sequentially displaced to and operated at two or more ablation sites during a surgical procedure, or by utilizing up to three independent cryoprobes inserted simultaneously in a uterus, for example, one in the uterine cavity and one in each of the cornua, and using sonography to confirm that the cryosurgical devices are properly positioned in the uterine cavity and to monitor the growth of the ice crystal during the treatment cycles. Cryoablation techniques also include using a coolable balloon operable to cool the endometrium. This procedure includes inserting a balloon into the uterine cavity, filling the balloon to the proper size and cooling the balloon to freeze the endometrial tissue. Freezing of tissue with a cryoprobe may result in tearing of the tissue that may be frozen to the device due to movement of the probe or patient. This may result in complications such as excessive bleeding during or after the procedure. This may occur even when attempts are made to free the device from the tissue by warming the cryoprobe before removal or other preventive procedures.

In addition, when treating targeted endometrial tissue, there is a trade-off between the options of effectively inactivating the tissue intended for removal while minimizing unavoidable damage to the patient's nerves or organs (e.g. bladder, rectum and ovaries) adjacent to such tissues.

To summarize, it has been seen that some prior art techniques are highly invasive, that other prior art techniques are less highly invasive but are likely to entail surgical complications and require highly skilled operators with specialized training. Therefore there is a widely felt need for, and it would be highly advantageous to have, an apparatus and technique for endometrial ablation which is minimally invasive and does not require highly skilled operators and specialized training.

It has also been seen that most endometrial ablation procedures are painful. In particular, most such procedures require anesthesia during performance of the ablative process, and therefore are of limited applicability in simpler clinical settings. In general, there is a widely felt need for, and it would be highly advantageous to have, an apparatus and technique for endometrial ablation that minimizes bleeding and the risk of damaging adjacent tissue, which is not painful, does not require anesthesia during treatment, and which therefore is appropriate for use in an “office visit” setting.

### BRIEF SUMMARY OF THE INVENTION

The present invention is directed towards apparatuses and methods for the selective ablation of unwanted tissue. The invention is particularly applicable to the ablation of endometrial tissue for the treatment of menorrhagia. Delivering ultrasonic and cryogenic energies simultaneously and/or sequentially, the present invention may be used to destroy and/or remove unwanted and diseased tissue. Combining the delivery of ultrasonic and cryogenic energy during treatment, the present invention may provide advantages over existing methods and devices for removing unwanted and/or diseased tissue.

The apparatus in accordance with the present invention may be embodied as a hand held device and may comprise a body having a proximate end and a distal end. The proximate end may also include a handle. The distal end may comprise an ultrasonic tip. The body may define one or more chambers. Delivering a cryogenic fluid, such as, but not limited to, a liquid or gas, into one or more of the chambers may cool the ultrasonic tip. Transferring thermal energy away from the tissue, the ultrasonic tip when sufficiently cooled and placed proximate to the tissue may be used to ablate unwanted and/or diseased by freezing the tissue. Causing the tip to vibrate at an ultrasonic frequency, an ultrasonic transducer mechanically connected to the ultrasonic tip may be used to excite the ultrasonic tip and keep it free from the frozen tissue. Targeting selected tissue, the present invention removes diseased and/or unwanted tissue without unduly damaging healthy tissues surround the targeted tissue. The cryogenic fluid may be delivered to the chambers of the ultrasonic tip's body through one or more interior passages or similar elements. Acting as inlets, the interior passages introduce cryogenic fluid from a reservoir into a chamber. Acting as outlets, the interior passages permit cryogenic fluid to flow through and/or out of a chamber. A chamber may be designed to approach the distal end of the ultrasonic tip. Exciting the distal end of the ultrasonic tip may enable the transmission of ultrasonic energy to a tissue. The transmission of ultrasonic energy to a tissue may occur during, before, and/or after the transfer of thermal energy away from the tissue. The transmission of ultrasonic energy and/or the transfer of thermal energy may occur through direct contact of the ultrasonic tip with the tissue. Alternatively, an accumulation of frost on the tissue and/or the ultrasonic tip may act as a conduit for the transfer of thermal energy and/or transmission of ultrasonic energy.

Freezing of the uterine lining is also useful for endometrial ablation. Cryosurgical procedures involve deep tissue freezing which results in tissue destruction due to rupture of cells and or cell organelles within the tissue. Deep tissue freezing is effected by insertion of a tip of a cryosurgical device into the tissue, either abdominably, endoscopically or laparoscopically, and a formation of an ice-ball around the tip.

With conventional cryotherapy, in order to effectively destroy a tissue there is a need to locate the isothermal surface of −40 degree C. at the periphery of the treated tissue, thereby exposing adjacent, usually healthy, tissues to the external portions of the ice-ball. The application of temperatures of between about −40 degree C. and 0 degree C. to such healthy tissues usually causes substantial damage thereto, potentially resulting in temporary or permanent impairment of functional organs.

Under the present invention it is possible to simultaneously and/or sequentially apply ultrasonic and cryogenic treatments to further control and direct treatment. This allows for destruction of treated tissues at temperatures significantly above −40 degree C. with the application of ultrasound and reduces the risk to nearby functional organs and tissues.

Another advantage of the present invention may be avoiding the adhesion of the ultrasound tip to the tissue during cryogenic ablation due to the ultrasonic vibration of the ultrasound tip.

Another advantage may be that the vibration created by the ultrasonic energy delivered to the tissue separates the unwanted and/or diseased tissue from healthy and/or wanted tissue.

Another advantage may be the destruction of microorganisms in the treatments by the delivered ultrasonic and/or cryogenic energy.

Another advantage may be the creation of an analgesic effect providing inherent pain relief on the treated tissue by the delivered ultrasonic energy.

A further advantage of the invention may be the delivering ultrasonic energy before, during, and/or after ablation to decrease healing time and/or provide other positive benefits to the surviving tissue.

Another advantage of the invention may be the selective destruction of the frozen tissue by matching the ultrasound vibrations to the resonant frequencies of the frozen tissue which may be different than the resonant frequencies of the normal tissues.

### BRIEF DESCRIPTION OF THE SEVERAL VIEWS OF THE DRAWING

FIG. 1 depicts a dimensional view of an embodiment of the apparatus according to the present invention.

FIG. 2 depicts a cross-sectional view of an embodiment of the hand held portion of the device.

FIG. 3 depicts a top cross-sectional view of an alternative embodiment of the hand held portion of the device.

FIG. 4 depicts a cross-sectional view of the distal end of an alternative embodiment of the ultrasound tip.

FIG. 5 shows a cross-sectional view of the distal end of an alternative embodiment of the ultrasound tip with a frost layer.

FIG. 6 depicts a cross-sectional view of the distal end of an alternative embodiment of the ultrasound tip.

FIGS. 7A-7E depicts cross-sectional views of various alternative embodiments of the distal end of the ultrasound tip.

FIGS. 8A-8E depicts cross-sectional views of various alternative embodiments of the distal end of the ultrasound tip.

FIG. 9 depicts a cross-sectional view of the distal end of an alternative embodiment of the ultrasound tip.

FIG. 10 depicts a cross-sectional view of the distal end of an alternative embodiment of the ultrasound tip having an expandable balloon.

### DETAILED DESCRIPTION OF THE INVENTION

The present invention relates to devices and methods for the combination of use of ultrasonic energy and cryogenic cooling for tissue ablation. The invention is particularly applicable for endometrial ablation for the treatment of menorrhagia. Highly controllable, precise delivery of ultrasonic energy and cryogenic cooling allows precise destruction of endometrial tissue while minimizing damage to surrounding tissue. The ultrasonic energy and cryogenic cooling may be delivered simultaneously and/or sequentially to the tissue being ablated. The combination of ultrasonic energy treatments provides a synergistic effect that allows effective treatment at higher temperatures than is possible with cryogenic cooling alone. Although cryogenic cooling may be herein referred to as the delivery of energy, it is of course the transfer of thermal energy from the tissue to the device that results in the cooling of the tissue. Of course, the cryogenic cooling is also used to remove the heat generated by the application of the ultrasound energy. In addition to its preferred use for endometrial tissue ablation, the device can be used for tissue ablation generally on human patients and for veterinary uses.

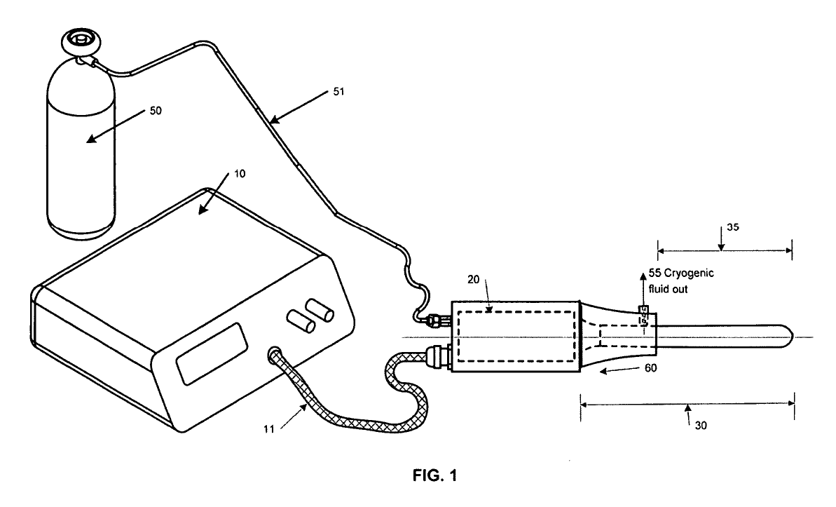

FIGS. 1-10 relate generally to describe the invention in some of the aspects of its embodiments. With respect to FIG. 1, a general view of an embodiment of the apparatus is shown. The apparatus of the present invention may be a hand held device with a housing 60 surrounding an ultrasound transducer 20 as shown in FIG. 1. The housing 60 provides a surface for the surgeon to hold for manipulation of the device over the wound. The housing 60 also may provide dampening and isolation so that the heat, electrical and mechanical energy emitted from the ultrasound transducer 20 do not interfere with the operator's control of the device. The housing 60 may extend over a portion of the ultrasound transducer tip 30 to insulate the ultrasound transducer tip 30 and isolate portions of the proximal end of the ultrasound transducer tip 30 from contact with endometrial tissue.

The ultrasound transducer 20 is driven by an ultrasound generator 10. The ultrasound generator 10 is typically powered with standard AC current which is electrically connected to an ultrasound transducer 20 through a cable 11 and activated with a hand or foot operated switch. The ultrasonic transducer 20 is pulsed according to a driving signal generated by the ultrasound generator 10 and transmitted to the ultrasonic transducer 20 by cable 11. The driving signals, as a function of time, may be rectangular, trapezoidal, sinusoidal, triangular or other signal types as would be recognized by those skilled in the art.

The ultrasound generator 10 may also be programmable to provide a rapid pulsed on-off signal to the ultrasound transducer 20 to modify the vibrational interaction between the transducer tip 30 and the tissue 80 which may control and limit friction, tissue attachment, standing wave production and temperature rise within the tissue. This pulsed signal may vary between 0 to 100% depending on the application.

The distal end of the ultrasound transducer 20 is attached to a transducer tip 30 for conditioning and directing the ultrasonic energy toward the tissue area selected for treatment. The ultrasound waves are emitted at a frequency and amplitude. The ultrasonic frequency may be used in embodiments that include low frequency or high frequency embodiments that operate within the range of 15 kHz and 20 mHz. The preferred frequency range for the transducer tip 30 is 15 kHz to 50 kHz with a recommenced frequency of approximately 30 kHz.

The amplitude of the ultrasonic waves may be between 1 micron and 250 microns with a preferred amplitude in the range of 10 to 50 microns and a recommended amplitude of 20 microns.

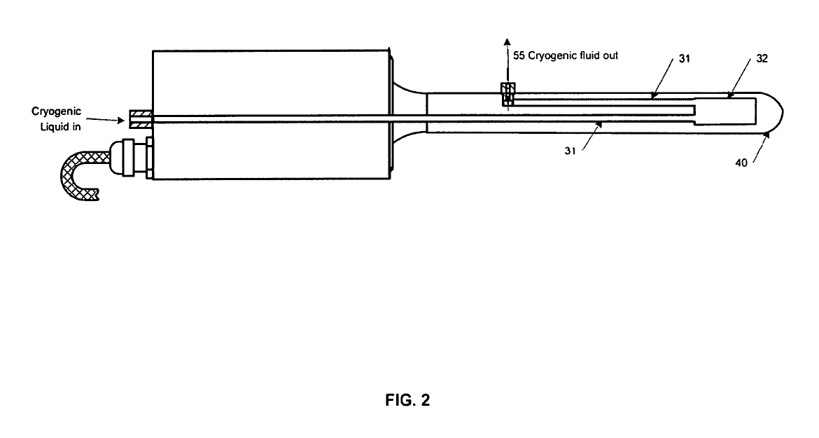

Cryogenic cooling may be utilized to cool the transducer tip 30 and adjacent tissue 80. The ultrasound transducer tip 30 may contain one or more interior passages 31 for transfer of a cryogenic fluid 55 through the transducer tip 30. The delivery of cryogenic fluid 55 may be simultaneous or sequentially to the delivery of ultrasonic energy. The cryogenic fluid 55 is also used to remove heat generated from the ultrasound energy within the transducer tip 30. The interior passage 31 through which cryogenic fluid 55 flows through the transducer tip 30 may include an expansion shown as a chamber portion 32 in FIG. 2.

A cryogenic source 50 may be used to supply the cryogenic fluid 55. One or more delivery tubes 51 are typically used to deliver the cryogenic fluid 55 from the cryogenic source to the transducer tip 30. The cryogenic source 50 may include a refrigeration system that recycles the cryogenic fluid 55 through the transducer tip 30 or it may be vented once-through system such as those using liquid nitrogen or liquid carbon dioxide.

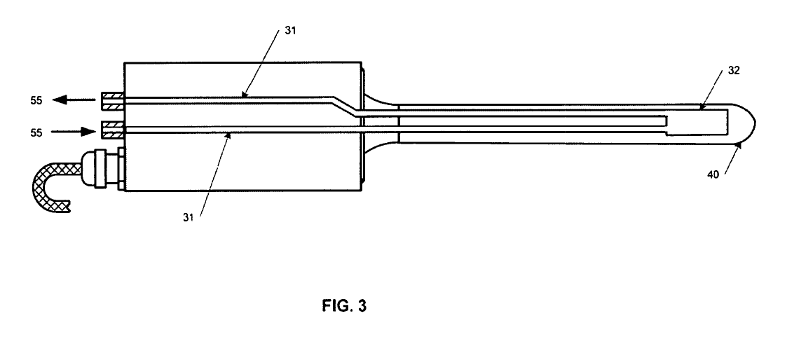

A cryogenic source 50 may be a refrigeration system capable of recycling the cryogenic fluid 55 through the transducer tip 30. The interior passage 31 layout of FIG. 3 may be preferred for use with a refrigeration system. Typical examples include liquid/vapor compression type units that utilize a condensation-evaporation cycle or Joule-Thompson type refrigeration systems. Joule-Thompson refrigeration systems utilize a pressurized gas that cools when decompressed such as, but not limited to, argon, air, carbon tetra-fluoride, xenon, krypton, nitrous oxide or carbon dioxide. The gas used as a cryogenic fluid 55 is pressurized and then decompressed in an expansion chamber such as a chamber portion 32 resulting in cooling of the gas within the transducer tip 30.

The ultrasonic tip 30 may also contain one or more temperature sensors which may control the flow rate of cryogenic fluid 55 through the ultrasound transducer tip 30 to maintain a constant preselected temperature at the tip regardless of the ultrasound energy emitted from the tip. A temperature controller may also be used to vary the temperature through a manually controllable or a preprogrammed cycle. The ultrasound tip 30 may then be placed adjacent to the tissue 80 to be ablated to create an area of frozen tissue 85 as shown in FIG. 4.

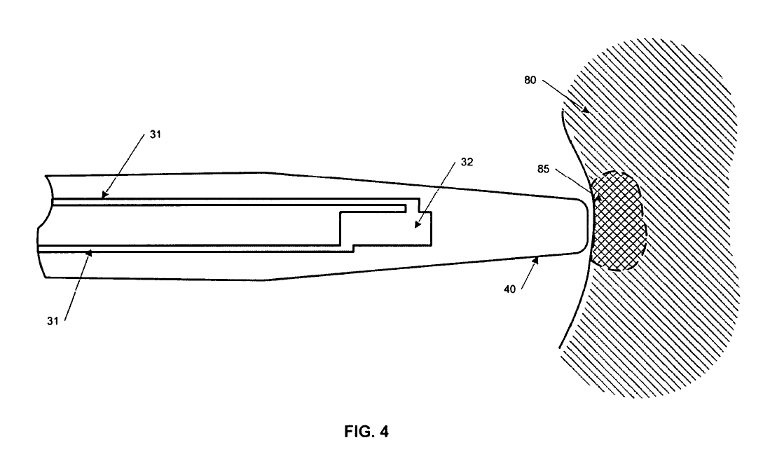

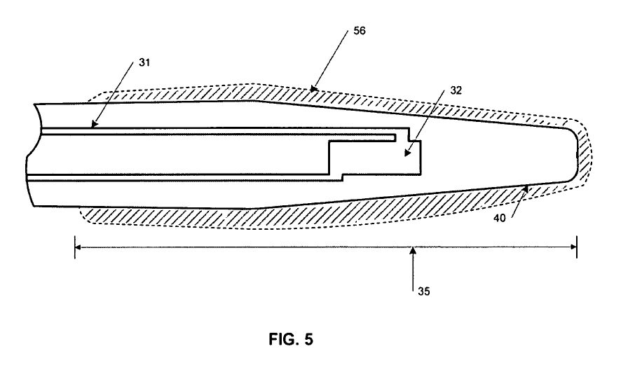

The ultrasonic tip 30 provides an ultrasonically active distal end 35 as well as cryogenic energy to tissue 80 through direct contact or indirectly through an accumulation as frost 56 as shown in FIG. 5. Creating the accumulation of frost 56 on distal end 35 of the ultrasonic tip 30 may be accomplished by allowing moisture from the air to condense and freeze on distal end 35. Alternatively, an accumulation of frost 56 may be formed with a substance such as, but not limited to, water being placed on distal end 35 and allowed to freeze.

Cryosurgical procedures involve deep tissue freezing which results in tissue destruction due to rupture of cells and/or cell organelles within the tissue 80. Deep tissue freezing is achieved by insertion of a tip of a cryosurgical device into the tissue 80, either vaginally, endoscopically or laparoscopically, and a formation of an ice-ball around the tip from the tissue to be removed.

In order to effectively destroy tissue by such an ice-ball, the diameter of the ball should be substantially larger than the region of tissue 80 to be treated, a constraint derived from the specific profile of temperature distribution across the ice-ball.

Specifically, the temperature required for effectively destroying a tissue without the application of ultrasound is about −40 degree C. or cooler. However, the temperature at the surface of the ice-ball is 0 degree C. The temperature declines exponentially towards the center of the ball such that an isothermal surface of about −40 degree C. is typically located within the ice-ball substantially at the half way point between the center of the ball and its surface.

Thus, in order to effectively destroy a tissue by freezing alone, without application of ultrasound, there is a need to locate the isothermal surface of −40 degree. C. at the periphery of the treated tissue, thereby exposing adjacent, usually healthy, tissues to the external portions of the ice-ball. The application of temperatures of between about −40 C. and 0 degree C. to such healthy tissues usually causes substantial damage thereto, potentially resulting in temporary or permanent impairment of functional organs.

In addition, given the geometry of endometrial tissue, when the adjacent tissues 80 are present at unsymmetrical positions with respect to the frozen tissue 85, and since the growth of the ice-ball is in substantially similar rate in all directions toward its periphery, if the tip of the cryosurgical device is not precisely centered, the ice-ball may reach healthy tissue 80 before it reaches the tissue to be treated, and decision making of whether to continue the process of freezing, risking a damage to adjacent healthy tissues, or to halt the process of freezing, risking a non-complete destruction of the treated tissue, must be made.

Although the present invention is applicable to any cryosurgical treatment, discussion is hereinafter primarily focused on a cryosurgical treatment of an endometrium. Thus, when performing endometrial ablation, there is a trade-off between effectively destroying the tissue selected for ablation and causing unavoidable damage to the patient's adjacent tissues and organs such as ovaries, urethra, bladder, rectum and nerves. Endometrial ablation may not require total destruction of the entire volume of tissue as does treatment of a malignancy, nevertheless it does run the risk of causing damage to healthy functional tissues and adjacent organs if care is not taken to limit the scope of destructive freezing to appropriate locations.

The application of ultrasonic energy itself makes the treated area less painful due to the pain relief provided by the application of the ultrasonic energy to nerve endings associated with the wound area. The shape of the radiation surface can be modified to optimize this effect.

Under the preferred embodiment, the cryogenic cooling of the ultrasound tip also has therapeutic value associated with wound treatment. The cryogenic fluid 55 used to cool the transducer tip 30 will also cool the surface of the wound. Cooling an incision wound is common practice to reduce the edema, pain, swelling and/or inflammation associated with wound treatment.

Due to the synergistic impact of the ultrasound treatment with the cryogenic treatment of the present invention, it is much easier to avoid the loss of healthy tissue while performing the ablation. This occurs because it is not necessary to lower the temperature of the tissue being treated to −40 degrees C. to destroy the treated tissue when ultrasound is being applied. This feature avoids damage to healthy tissues. Furthermore, the ultrasound energy is highly controllable and may be applied in procedures customized for each patient and situation. Furthermore, the use of ultrasound energy may also be customized to utilize the differences in resonant frequencies between the frozen tissue and tissues not frozen to resonate the ultrasonic vibrations with tissue cells and elements of tissue cells to maximize disruption of frozen tissues and minimize impacts oil tissues that may not be frozen or may be of other tissue types. Thus the synergism between the cryogenic energy and ultrasonic energy can be utilized to minimize negative effects.

Further synergisms are achieved between the ultrasonic energy and cryogenic energy by utilizing the ultrasonic vibrations to keep the ultrasound tip 30 free from the frozen tissue as the frozen tissue is formed. Ultrasonic vibrations generate heat within the ultrasound tip 30 due to the energy dissipation, as well as between the ultrasound tip and the tissue due to friction between the vibrating ultrasound tip and the adjacent tissue. If needed, the ultrasonic energy may also be utilized to warm the ultrasound tip 30 and immediately surrounding frozen tissue 85 by interrupting or terminating cryogenic cooling.

The ultrasound energy emitted from the ultrasound transducer 20 may have a radial wave component and a lateral or longitudinal wave component. The magnitudes of the components are a function of the ultrasound transducer 20 used, the characteristics of the signal driver and the characteristics of the ultrasound tip 30. As shown in, but not limited to, the attached figures, a variety of geometries are available for use for the ultrasound tip 30. Specific features of the ultrasound tip 30 may be placed at locations along the axial length of the ultrasound tip corresponding with node and antinode positions of the ultrasound waves. For example, to minimize vibration, it may be desirable to locate the delivery tube 51 connection at a node point with no vibrational amplitude. Additionally, it may be desirable to minimize tissue freezing to the device by locating the ultrasound tip distal end at an antinode point of maximum vibration.

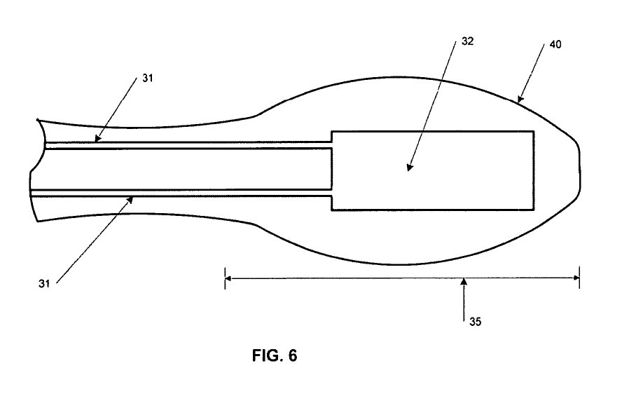

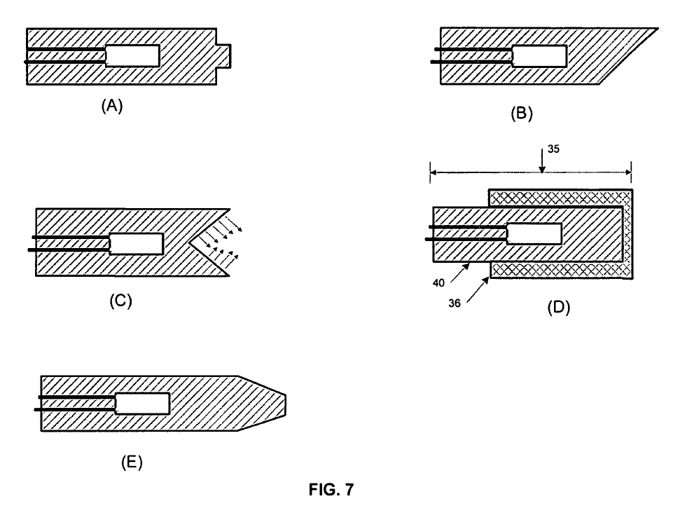

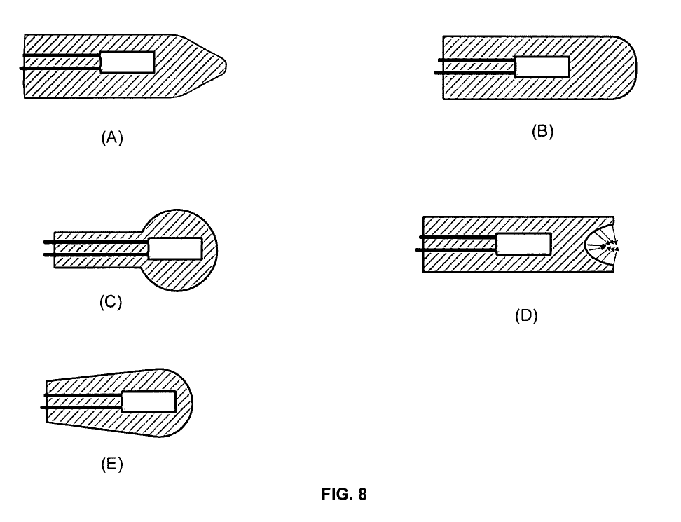

Choosing the geometry of the distal end of the ultrasound transducer tip 30 may have a significant impact on the relative magnitudes of the radial wave component size and the lateral or longitudinal wave component size. Available ultrasound tip geometries include oval, flat, curved, concave, ellipsoid, rounded or oval distal ends. The embodiment depicted in FIG. 6 may be useful for the ablation of large regions of tissue. In other aspects, the distal end of the ultrasonic tip may have various sizes and geometric shapes of the radiation surface 40 such as flat, concave, convex, rounded and/or angled. Some of these embodiments are shown in FIGS. 7 and 8. FIGS. 7C and 8D show various embodiments of the radiation surface 40 that will concentrate or focus the ultrasound energy. FIG. 7D shows a cylindrical shaped ultrasound tip 30 that may be particularly useful for minimizing radial wave components. An ellipsoid shaped ultrasound tip as shown in FIG. 6 may be useful for maximizing radial wave components along certain portions of the ultrasound tip 30. A cone distal end, depicted in FIG. 8A, may be useful the precise ablation of small regions of tissue. As shown in FIG. 7D, a disposable cover 36 matching the geometric conformation the of distal end may be used to cover the distal end of the ultrasound tip 30.

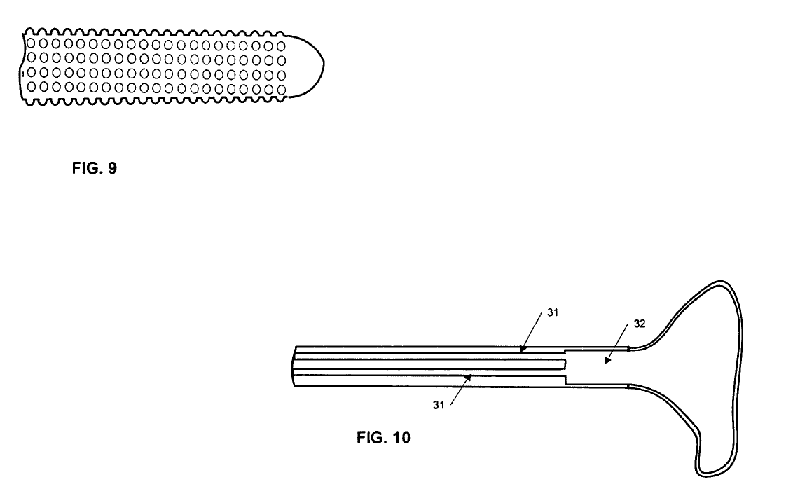

The surface of the ultrasound tip may be smooth or have various roughness features to increase abrasion or reduce surface contact with the tissue 80. FIG. 9 presents an alternative embodiment showing round protrusions over the surface of ultrasonic tip 30. Other possible roughness features may include waves, ridges, pins, cones or random granular elements of various sizes. A detachable ultrasound transducer tip 30 can allow a surgeon to vary the geometric shape of the distal end as appropriate either between procedures or during the course of a procedure.

Another embodiment of the present invention includes an ultrasound tip 30 that includes an inflatable distal end as shown in FIG. 10. In this embodiment, the balloon end may be a flexible membrane that is compact for insertion and positioning and may be folded within the ultrasound tip 30. The flexible membrane is fillable with a fluid when positioned. The fluid may be a liquid or a gas, preferably cryogenic fluid 55 may be used to fill the flexible membrane. This embodiment as depicted may be particularly useful for the ablation of large regions without repositioning the device. A triangular shape may be particularly useful that would approximately correspond with the shape of the endometrial tissue being ablated. The membrane may be constructed of a material that is fillable to a substantially predetermined fixed size and shape, or a membrane that may be of an adjustable size changeable by varying the fluid pressure within the balloon based on patient requirements.

Those skilled in the art will recognize that the ultrasound tip 30 can be a single piece unit or composed of one or more individual separate pieces that are detachable from the device. This allows interchangeability of portions of different embodiments of the tip as well as easier cleaning/sterilization of portions of the device and/or allows construction of disposable single-use portions of the ultrasound transducer tip 30. The transducer tip 30 is typically made from a metal such as alloys of titanium, aluminum and/or stainless steel. The portions of the ultrasound transducer tip 30 may also be made from plastic for disposable single-use embodiments of selected portions or protective coverings of the transducer tip 30.

The ultrasound tip 30 may be coated to further enhance the ability of the ultrasound tip 30 to remain free within the frozen tissue 85. For example, fluorocarbons, titanium nitride, polyvinylidene fluoride or poly(tetrafluoroethylene) are examples for materials that would be recognized by those skilled in the art for coating a cryogenic ultrasound tip useful with the present invention.

A method in accordance with the present invention comprises the steps of transmitting ultrasonic energy to and transferring thermal energy from a tissue to be ablated. The transfer of thermal energy from the tissue may proceed, follow, and/or occur simultaneously with the transmission of ultrasonic energy to tissue. Transferring thermal energy from the tissue may be accomplished by providing cryogenic fluid 55 to distal end 35 and placing distal end 35 proximate to and/or in contact with the tissue to be ablated. Transmitting ultrasonic energy to the tissue may be accomplished by exciting distal end 35 by activating transducer 20 and placing distal end 35 proximate to and/or in contact with the tissue. When ultrasonic energy and cryogenic energy are simultaneous delivered to the frozen tissue 85, the distal end 35 should remain proximate to and/or in contact with the tissue until the unwanted tissue is ablated. Alternatively, cryogenic energy and the ultrasonic energy may be applied sequentially to the tissue to be ablated. For example, a sequential application may begin by exciting distal end 35, placing the distal end 35 proximate to and/or in contact with the frozen tissue 85, and then providing cryogenic fluid 55 to the distal end 35. The sequence may be modified so that the sequence would begin by providing cryogenic fluid 55 to the distal end 35, allowing an accumulation of frost 56 to form on the distal end 35, placing the distal end 35 proximate to and/or in contact with the frozen tissue 85, and then activating the ultrasonic transducer. Sequential application of ultrasonic energy without providing cryogenic fluid 55 may result in warming of the distal end 35 to facilitate removal of the device.

The application of ultrasonic energy may have an antimicrobial effect for the treated and surrounding tissue. The application of ultrasound energy is known to produce cellular disruption and microbial inactivation due to cavitation in gases, liquids and/or tissues to which it is applied. The cavitations and the ultrasound energy are able to inactivate microbes in the area of treatment through cellular disruption, denaturization and other means. This effect can reduce the chance of infection, thereby greatly enhancing patient recovery, since post-surgical infection can be a major impediment to optimal patient recovery.

It should be appreciated that as used herein the delivery of cryogenic energy to a tissue is synonymous with transferring thermal energy away from a tissue. It should also be appreciated that as used herein the delivery of ultrasonic energy to a tissue is synonymous with the transmission of ultrasonic energy to a tissue. It should also be appreciated that as used herein the delivery of cryogenic energy to a tissue is synonymous with transferring heat away from a tissue to lower tissue temperature. It should be further appreciated that as used herein the delivery of ultrasonic energy to a tissue is synonymous with the transmission of ultrasonic energy to a tissue. It should be appreciated that elements described with singular articles such as “a”, “an”, and “the” or otherwise described singularly may be used in plurality. It should also be appreciated that elements described in plurality may be used singularly. Although specific embodiments of apparatuses and methods have been illustrated and described herein, it will be appreciated by people of ordinary skill in the art any arrangement, combination, and/or sequence that is calculated to achieve the same purpose may be substituted for the specific embodiments shown. It is to be understood that above description is intended to be illustrative and not restrictive. Combinations of the above embodiments and other embodiments as wells as combinations and sequences of the above methods and other methods of use will be apparent to individuals possessing skill in the art upon review of the present disclosure. The scope of the present invention should be determined with reference to the appended claims, along with the full scope of equivalents to which such claims are entitled.

I claim:

1. A method for endometrial cryoablation comprising the steps of:
providing an ultrasound generator for driving an ultrasound transducer;
vibrating a transducer tip attached to the ultrasound transducer;
passing a cryogenic fluid through an interior passage within the transducer tip;
freezing the tissue at a temperature below zero degrees centigrade;
emitting ultrasound waves from a radiation surface on the transducer tip; and
ablating the tissue through synergistic cooperation between the ultrasound energy and the frozen tissue.

2. The method of claim 1 having the additional step of expanding a cryogenic fluid within a chamber portion of the interior passage.

3. The method of claim 1 wherein the radiation surface is disposable.

4. The method of claim 1 wherein the ultrasound energy is generated from a signal form selected from the group including sinusoidal, rectangular, trapezoidal or triangular.

5. The method of claim 1 wherein the ultrasound energy is pulsed.

6. The method of claim 1 having the additional step of directing the radiation surface by holding a housing fitted over the ultrasound transducer.

7. The method of claim 1 wherein the radiation surface emits ultrasound waves at a wavelength between 16 kHz and 20 mHz.

8. The method of claim 1 wherein the radiation surface emits ultrasound waves at an amplitude between 1 micron and 250 microns.

9. The method of claim 1 having the additional step of providing pain relief with the ultrasonic energy.

10. The method of claim 1 wherein the ultrasound waves are emitted at approximately the resonant frequency of the frozen tissue.

11. The method of claim 1 having the additional step of an expandable portion of the transducer tip to form a balloon shaped radiation surface.

12. The method of claim 1 having the additional step of warming the transducer tip by terminating application of cryogenic fluid.

13. The method of claim 1 having the additional step of forming a layer of frost over the transducer tip before ablating the tissue.

14. The method of claim 1 having the additional step of focusing the ultrasound energy to a point.

15. The method of claim 1 having the additional step of deactivating microbes with the ultrasonic energy.

16. The method of claim 1 wherein the ultrasound waves from the radiation surface have a longitudinal component and a radial component.

17. The method of claim 1 wherein the radiation surface includes a coating.

18. The method of claim 1 having the additional step of measuring the temperature of the ultrasound tip with a temperature sensor.

19. The method of claim 18 having the additional step of controlling the emission of ultrasound waves and the passing of the cryogenic fluid with a controller.

20. The method of claim 1 wherein the step of emitting the ultrasound waves from the radiation surface also maintains the frozen tissue free from the ultrasound tip during the ablation of the tissue.
